(19) United States
(12) Patent Application Publication
(10) Pub. No.: US 2024/0166498 A1
(43) Pub. Date: May 23, 2024

(54) RESPONSIVE PLATFORM, CELLULAR DELIVERY KIT AND CELLULAR DELIVERY METHOD

(71) Applicant: Academia Sinica, Taipei City (TW)

(72) Inventors: Hsiao-hua YU, Taipei City (TW); Hsien-Ming LEE, New Taipei City (TW); Bhaskarchand Sureshchand Gautam, Hsinchu City (TW)

(21) Appl. No.: 17/982,383

(22) Filed: Nov. 7, 2022

(51) Int. Cl. B81C 1/00; A61K 47/69; B82Y 40/00

(52) U.S. Cl. CPC B81C 1/00111; A61K 47/6911; B82Y 40/00; B81B 2203/0361

(57) ABSTRACT

Disclosed is a responsive platform, which includes a polymer-grafted nanopillar array, cargo-containing entities, first conjugatable moieties, and second conjugatable moieties. The polymer-grafted nanopillar array includes thermoresponsive polymer brushes grafted onto surfaces of nanopillars, and the cargo-containing entities are attached to the thermoresponsive polymer brushes through non-covalent association between the first conjugatable moieties and the second conjugatable moieties. Accordingly, the cargo-containing entities can be released from the nanopillar array for cellular uptake in a controlled manner by applying thermal stimulus.

### BACKGROUND OF THE INVENTION

### 1. Field of the Invention

The present invention relates to a responsive platform and, more particularly, to a responsive platform with thermoresponsive polymer brushes, a cellular delivery kit and a cellular delivery method using the same.

### 2. Description of Related Art

An efficient and generalized route for in vitro biomolecules delivery is on constant demand for cell reprogramming and tissue engineering. Nanoinjection approaches can be used to deliver a diverse range of biomolecules in a controllable manner. However, this conventional nanoinjection technique can only deliver its cargo to one cell at a time, which makes this method ineffective for a large number of cells. High aspect ratio nanomaterials (nanopillars) assisted platform can be used for biomolecules delivery to a large number of adhered cells in a single time.

To date, only few attempts have been made to developed nanopillars for biomolecules delivery in vitro. Shalek.et.al used low-density vertical silicon nanowires to demonstrate DNA, siRNA, peptide and protein delivery into the cell interior though membrane penetration. However, these low-density nanowires not only provide a lesser amount of cargo loading but also rely on cell penetration for the delivery to the adhered cells. These low-density nanosurfaces frequently cause cell rupturing or apoptosis. Thus, further modifications are required in order to make these nano-based platforms more compatible for biological applications.

### SUMMARY OF THE INVENTION

Described herein are systems, compositions, kits and methods which can exhibit improved cell compatibility and controlled and efficient in-vitro deliveries. Specifically described are responsive platforms useful for control over the delivery of biomolecules into the cells tethered (adhered) on the platform.

One aspect of the disclosure provides a responsive platform, which includes a polymer-grafted nanopillar array, cargo-containing entities, first conjugatable moieties, and second conjugatable moieties. The polymer-grafted nanopillar array includes a nanopillar array and thermoresponsive polymer brushes grafted onto surfaces of nanopillars of the nanopillar array. The cargo-containing entities each include a cargo container and a cargo in the cargo container, and are attached to the thermoresponsive polymer brushes through non-covalent association between the first conjugatable moieties attached to the thermoresponsive polymer brushes and the second conjugatable moieties each attached to the cargo container of a respective one of the cargo-containing entities.

Another aspect of the disclosure provides a cellular delivery kit, which includes a conjugatable polymer-grafted nanopillar array and conjugatable cargo-containing entities. The conjugatable polymer-grafted nanopillar array includes a nanopillar array, thermoresponsive polymer brushes grafted onto surfaces of nanopillars of the nanopillar array, and first conjugatable moieties attached to the thermoresponsive polymer brushes. The conjugatable cargo-containing entities each include a cargo-containing entity and a second conjugatable moiety attached to a cargo container of the cargo-containing entity and configured to allow non-covalent association with a respective one of the first conjugatable moieties.

Yet another aspect of the disclosure provides a cellular delivery method, which includes steps of: seeding cells on the above-mentioned responsive platform, and applying thermal stimulus to the responsive platform to cause detachment of the cargo-containing entities from the thermoresponsive polymer brushes and to allow for uptake by the cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic depiction of an exemplary responsive platform.

FIG. 2 shows a schematic representation of the responsive nanosubstrate-mediated delivery.

FIG. 3 shows silicon nanosubstrate pillar (SiNP) surface analysis before and after biotinylated-poly(NIPAAm-co-AEMA) grafting: (a, b and c, d) water contact angle (WCA) before and after polymer coating at 37° C. and 4° C. respectively (insignificant variations in WCA were quantified before polymer coating, whereas, after polymer grafting the variation in WCA were immense at 37° C. and 4° C.); and (e, f and g, h) scanning electron microscope (SEM) images of before and after polymer grafting respectively (nanopillars morphology was investigated using SEM, and nanopillars aggregated morphology was observed after polymer grafting).

FIG. 4 shows Cryo-EM (transmission electron cryomicroscopy) images of (a) empty liposomes, (b) calcein-loaded liposomes, and (c) doxorubicin loaded liposomes (scale bar: 100 nm, inset Figures are images of liposome under daylight and U.V. light of 365 nm), in which calcein-loaded liposome resembles empty liposome in Cryo-EM, while doxorubicin-loaded liposome shows clear crystal-like structure inside, indicative of successful doxorubicin loading.

FIG. 5 shows (a, b) Cryo-EM images of doxorubicin-loaded liposomes tethered on nanopillars via biotin-streptavidin-biotin association, which confirmed the attachment of liposome (the enlarges micrographic images confirmed the existence of doxorubicin); and (c, d) comparison of laser scanning confocal microscopy (LSCM) images of responsive nanosubstrate before (c) and after (d) thermal-stimulated detachment of calcein-loaded liposomes, which confirmed the detachment of liposomes upon thermal stimulation (scale bar:1 μm).

FIG. 6 shows quantified fluorescence intensity of calcein loaded-liposomes of detached and remained liposome for specific (gray) and nonspecific (black) interaction with responsive nanosubstrate.

FIG. 7 shows quantitative measurements of cell viability for plain control (the upper curve) and nanosurface (the lower curve) over 0.5, 1, 2 and 4 h incubation.

FIG. 8 shows quantified fluorescence intensity measurements of 4 mM calcein-loaded liposomes uptake by human embryonic kidney 293T cells (HEK-293T) after 0.5 hr, 1 hr, 2 hrs and 4 hrs time points after initial seeding: the liposomes were either in solution with thermal stimulation (circle, solid line) and without thermal stimulation (circle, dashed line) or conjugated on the responsive nanosubstrate with thermal stimulation (triangle, solid line) and without thermal stimulation (triangle, dashed line). Cells not treated with liposome (hexagon) were used as control for background signal.

FIG. 9 shows (A) multiple cells and (B) single cell confocal fluorescence micrograph images of HEK-293T cells on responsive nanosubstrate after initial seeding and liposomes detachment and uptake upon thermal-stimulation (scale bar: 10 μm applicable to all the images).

FIG. 10 shows quantified cell viability information from fluorescence images of live/dead assay of Michigan cancer foundation-7 (MCF-7) cells incubated on doxorubicin-loaded liposome conjugated responsive nanosubstrate without thermal stimulus (the upper curve) and with thermal stimulus (the lower curve).

FIG. 11 shows confocal fluorescence images of the control and endocytosis inhibitor pre-treated HEK-293T cells incubated for 4 h after stimulus release of calcein-loaded liposome (localization of calcein-loaded liposome into cells is visualized with green fluorescence), in which different endocytosis inhibitors (chlorpromazine, sodium azide, genistein, nocodazole and wortamannin) were used, and R18 cell membrane staining was used in order to locate cells (red color) (scale bar: 50 μm applicable to all the images).

### DETAILED DESCRIPTION OF THE PREFERRED EMBODIMENT

Described herein are compositions, systems, kits, and methods useful for delivering cargo-containing entities from nanosubstrates into tethered cells in a controlled manner bypassing the endocytosis pathways. The compositions, systems, kits, and methods utilize switchable phase transition of thermoresponsive polymers to control the release of the cargo-containing entities. Upon release of the cargo-containing entities from the nanosubstrates by thermal stimulation, high local concentration of the cargo-containing entities between the cells and the nanosubstrates can drive the cellular uptake with non-endocytic pathways.

### Definitions

The term “responsive” refers to materials displaying alteration of their intrinsic properties when subjected to external stimuli such as light, temperature, electricity, pressure, voltage and pH.

The terms “thermoresponsive” and “thermosensitive” refer to materials changing their shape or deform when subjected to temperature gradient. Poly(N-isopropylacryl amide) and poly(N-vinylcaprolactume) are some typical examples of thermosensitive materials.

The terms “repeat unit” and “monomeric unit” refer to structural repeat unit, which is minimum structural unit. The repeating of described structural unit constitutes regular macromole, regular oligomer molecules, regular block or rule chain.

The term “binding” refers to a first moiety interacting with a second moiety, wherein the first and second moieties can be in contact with one another.

The term “biotin-based” refers to a moiety including biotin and variations of biotin, such as biotin or desthiobiotin with an open ring or substitutions. Typically, a biotin and its derivative can show a highly specific interaction with a biotin-binding entity or protein, such as avidin, NeutrAvidin, or streptavidin.

The term “conjugate” refers to a process by which two or more molecules specifically interact. For instance, a biotin and a streptavidin conjugate.

The term “conjugatable” refers to a molecule that can specifically come together with another molecule to which it can be conjugated. For instance, a biotin is conjugatable to a streptavidin.

The term “instructional material” includes a publication, a recording, a diagram, a link, or any other medium of expression which can be used to communicate the usefulness of one or more compositions of the invention for its designated use. The instructional material of a kit of the invention may, for example, be affixed to a container which contains the composition or components or be shipped together with a container which contains the composition or components. Alternatively, the instructional material may be shipped separately from a container with the intention that the instructional material and a composition or component be used cooperatively by the recipient.

The phrases “bound to”, “conjugated to”, “conjugated with”, “conjugatable to”, “conjugatable with”, and “attached to” refer to being directly or indirectly bound/conjugated/attached. For instance, the cargo-containing entity can be indirectly bound to the thermoresponsive polymer brushes by the first conjugatable moiety and the second conjugatable moiety.

### Compositions

Described herein are compositions of matter including responsive platforms. The responsive platform can be used for e.g. biomolecule delivery in a controllable manner.

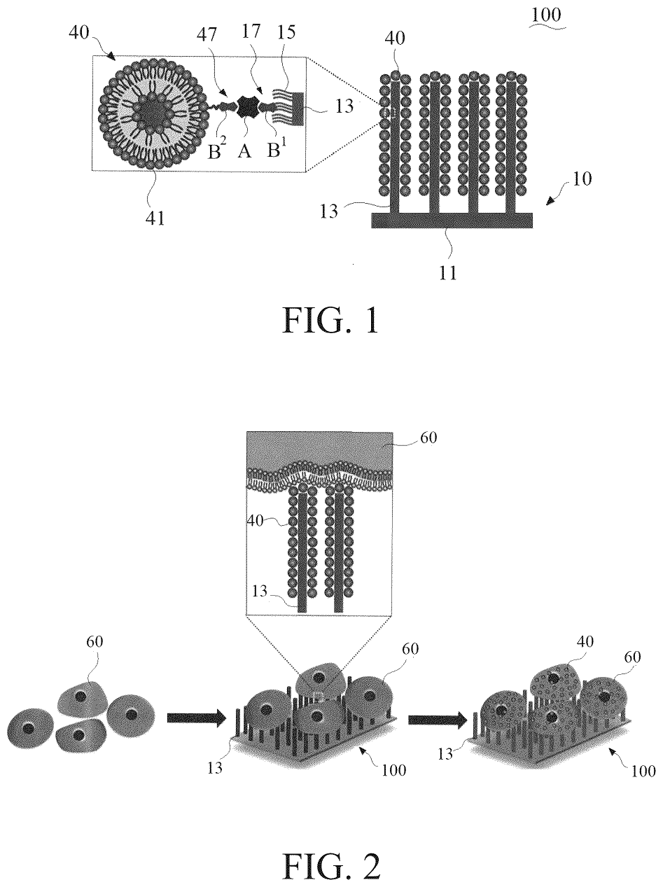

FIG. 1 shows a schematic depiction of a responsive platform 100. The right part of FIG. 1 schematically illustrates a polymer-grafted nanopillar array 10 (such as a polymer-grafted silicon nanopillar array) and cargo-containing entities 40 over surfaces of each nanopillar 13 of the polymer-grafted nanopillar array 10. In some embodiments, the polymer-grafted nanopillar array 10 can be prepared through photolithography and wet etching to form a plurality of nanopillars 13 on a base 11, followed by polymer grafting. The left part of FIG. 1 shows an expanded view of thermoresponsive polymer brushes 15 grafted onto surfaces of nanopillars 13 and the cargo-containing entities 40 each attached to a respective one of the thermoresponsive polymer brushes 15 through non-covalent association between a first conjugatable moiety 17 attached to the thermoresponsive polymer brush 15 and a second conjugatable moiety 47 attached to the cargo-containing entity 40. As a result, the association of the first conjugatable moiety 17 and the second conjugatable moiety 47 can form an affinity bridge which includes, for example, a receptor A and a first ligand B¹ and a second ligand B² both non-covalently conjugated with the receptor A. In this example, the cargo-containing entity 40 can include any type of cargo container 41 configured for loading a variety of cargos (such as drugs, dyes, biomolecules and the like) therein.

The responsive platform 100 illustrated in FIG. 1 can be prepared by the following exemplary steps: providing a conjugatable polymer-grafted nanopillar array with the polymer-grafted nanopillar array 10 and the first ligands B¹ covalently bonded to the thermoresponsive polymer brushes 15; providing the receptors A and non-covalently binding the receptors A to the first ligands B¹ with one-to-one non-covalent conjugation between the receptor A and the first ligand B¹; and providing conjugatable cargo-containing entities each with the second ligand B² covalently bonded to the cargo container 41 of the cargo-containing entity 40, and non-covalently binding the conjugatable cargo-containing entities to the receptors A with one-to-one non-covalent conjugation between the receptor A and the second ligand B².

FIG. 2 illustrates schematic representation of the responsive nanosubstrate-mediated delivery. After cells 60 are seeded on the responsive platform 100 as shown in FIG. 1, thermal stimulation induces hydrophobic-hydrophilic conversion of the thermoresponsive polymer brushes 15 (shown in the left part of FIG. 1) and thus causes detachment of the cargo-containing entities 40 from the surfaces of the nanopillars 13. In the restricted space between the cells 60 and the polymer-grafted nanopillar array 10, these detached cargo-containing entities 40 exist at a high local concentration. As a result, the cargo-containing entities 40 are uptaken by the cells 60 with high efficiency.

The new “nano on nano” platform technology can efficiently deliver, for example, liposomal content to cells in a relatively uniformly dose and patterned fashion, especially bypass the degradative endocytosis pathway. The platform has the following material features: (i) an array of nanopillars for creating a pseudo three-dimensional nano-environment for cell culturing, (ii) thermoresponsive polymer grafted onto nanopillars to form a responsive nanosubstrate, and (iii) immobilized cargo-containing entities using ligand-receptor recognition. The working principle is that the cargo-containing entities are detached for cellular uptake upon thermal stimulation and high local liposome concentration between the cells and the substrates drives the cellular uptake with non-endocytic pathways.

The thermoresponsive polymer brushes may be any thermosensitive material which can undergo hydrophobic—hydrophilic conversion when temperature gradient is applied. For example, the thermoresponsive polymer brushes may include polymer chains with “lower critical solution temperature (LCST)” behavior. Not-limiting examples of LCST polymers include poly (N-isopropylacrylamide) polymer (PNIPAAm, represented by the following formula (I-1)) and poly (N-vinyl caprolactam) (PNVCL, represented by the following the formula (I-2)):

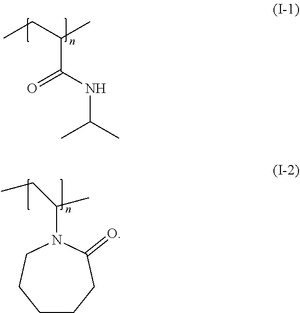

PNVCL and PNIPAAm polymers display similar LCST behavior in aqueous solutions with temperatures ranging between 30-32° C. which means that above this temperature the polymers show hydrophobicity and insolubility in water, and below LCST, they display hydrophility and high solubility in water. This reversible solubility behavior of the polymers in water is mainly caused by the hydrophilic and hydrophobic groups present in the structure. The mechanism of polymer reversible water solubility depends on hydrogen bonding between water molecules and monomeric units of the polymer. Partially charged groups (e g amine and carboxylic groups) of the polymer form hydrogen bonds with oxygen and hydrogen atoms of water molecules at low temperature. On the other hand, at higher temperature, polymer chains show contraction, thereby disabling water molecules to interact with the monomeric units of the polymer which results in polymer-water insolubility. Further, the thermoresponsive polymer brushes may be constructed from thermoresponsive co-ploymers. For instance, the PNIPAAm-based polymer grafting may involve copolymerization with hydrophilic monomers (e.g. amino ethyl methacrylate, AEMA)). Accordingly, in some specific embodiments, the nanopillars are coated by a polymerized layer of thermoresponsive co-polymer p(NIPAAm-co-AEMA) as the thermoresponsive polymer brushes, which includes a first repeat unit (monomeric unit) derived from a monomer of N-isopropyl acrylamide and represented by the following formula (I) and a second repeat unit (monomeric unit) derived from a monomer of amino ethyl methacrylate and represented by the following formula (II) (the symbol * being the attachment site for bonding to the first conjugatable moiety):

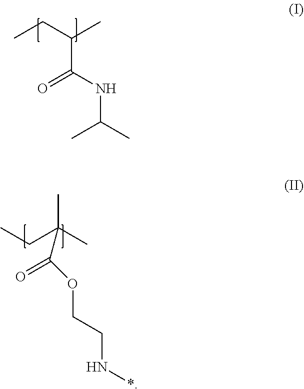

This p(NIPAAm-co-AEMA) coating is more compatible to cells (due to less cell penetration) and also provide temporal cargo detachment upon thermal stimulus.

To make the surface universal to nano injection all types of the delivery agent such as drugs, dyes, biomolecules and the like, liposomes can be employed as the cargo container (for its biocompatibility and capability to encapsulate versatile molecules) attached to the polymer-modified nanopillar surface (e.g. p(NIPAAm-co-AEMA)-SiNP surface) by, for example, B¹-A-B² bridge in which the A portion (i.e. the above-mentioned receptor) includes an avidin group, NeutrAvidin group, or streptavidin group, and the B¹ portion (i.e. the above-mentioned first ligand) and the B² portion (i.e. the above-mentioned second ligand) each independently include a biotin-based group. For instance, in some embodiments, liposomes are biotinylated by using one of the lipids consisting biotin group, and similarly, the nanopillars are also functionalized by biotin after polymer coating. Accordingly, having both the entity biotinylated streptavidin can be employed to attach the liposome through biotin-streptavidin-biotin (B¹-A-B²) association. Moreover, PEGylated and cholesterol lipids can also be used for the liposomal formulation to provide mechanical strength and to reduce steric hindrance on polymer-liposome and cell-liposome interface.

Methods

Also disclosed herein are methods of delivering agents (e.g. drugs, dyes, biomolecules and others) into cells. The methods may include the step of providing the above responsive platform, seeding cells on the responsive platform, and applying thermal stimulus to the responsive platform to cause detachment of the cargo-containing entities from the thermoresponsive polymer brushes and to allow for uptake by the cells. This method can generate much higher cargo-containing entity local concentration and exhibit higher cellular uptake compared with conventional solution-based incubation method for cellular uptake. After applying thermal stimulus, the cells can be incubated for a predetermined period for cellular uptake. To ensure cell viability, the predetermined period for cell incubation after thermal stimulus preferably is within 4 hours, e.g. 0.5 hour to 4 hours. Further, the cell incubation temperature may be 32° C. or greater, 33° C. or greater, 34° C. or greater, 35° C. or greater, 36° C. or greater, 37° C. or greater, 38° C. or greater, or 39° C. or greater. Preferably, the cell incubation temperature is within a range greater than 32° C. and less than 46° C., such as from 33° C. to 41° C., from 34° C. to 40° C., or from 35° C. to 39° C.

In some embodiments, said seeding the cells is performed at a first temperature at which the thermoresponsive polymer brushes exhibit contraction and hydrophobicity. Further, in some embodiments, said applying the thermal stimulus includes subjecting the thermoresponsive polymer brushes to a second temperature at which the thermoresponsive polymer brushes exhibit expansion and hydrophilicity. More specifically, for the nanopillar array modified with LCST-typed thermoresponsive polymer brushes, the first temperature is greater than the lower critical solution temperature of the thermoresponsive polymer brushes, and the second temperature is lower than the lower critical solution temperature of the thermoresponsive polymer brushes. For example, the first temperature may be 32° C. or greater, 33° C. or greater, 34° C. or greater, 35° C. or greater, 36° C. or greater, 37° C. or greater, 38° C. or greater, or 39° C. or greater, while the second temperature may be 24° C. or less, 22° C. or less, 20° C. or less, 18° C. or less, 16° C. or less, 14° C. or less, 12° C. or less, 10° C. or less, 8° C. or less, 6° C. or less, 4° C. or less, or 2° C. or less. Preferably, the first temperature is within a range greater than 32° C. and less than 46° C., such as from 33° C. to 41° C., from 34° C. to 40° C., or from 35° C. to 39° C., while the second temperature is within a range greater than 0° C. and less than 25° C., such as from 1° C. to 23° C., from 1° C. to 21° C., from 1° C. to 19° C., from 1° C. to 17° C., from 1° C. to 15° C., from 1° C. to 13° C., from 1° C. to 11° C., from 1° C. to 9° C., from 1° C. to 7° C., or from 1° C. to 5° C. Additionally, one or more times (e.g. four times) of thermo-released cycle (e.g. 4° C.-37° C.) can be employed for the thermal stimulation.

Said providing the responsive platform may include steps of: providing a conjugatable polymer-grafted nanopillar array having the polymer-grafted nanopillar array and the first conjugatable moieties; providing conjugatable cargo-containing entities each having the cargo-containing entity and the second conjugatable moiety; and attaching the cargo-containing entities to the thermoresponsive polymer brushes through the non-covalent association between the first conjugatable moieties and the second conjugatable moieties. In some embodiment, said attaching the cargo-containing entities to the thermoresponsive polymer brushes can be performed at an immobilization temperature at which the thermoresponsive polymer brushes exhibit contraction and hydrophobicity. More specifically, for the nanopillar array modified with LCST-typed thermoresponsive polymer brushes, the immobilization temperature is greater than the lower critical solution temperature of the thermoresponsive polymer brushes. For example, the immobilization temperature may be 32° C. or greater, 33° C. or greater, 34° C. or greater, 35° C. or greater, 36° C. or greater, 37° C. or greater, 38° C. or greater, or 39° C. or greater. Preferably, the immobilization temperature is within a range greater than 32° C. and less than 46° C., such as from 33° C. to 41° C., from 34° C. to 40° C., or from 35° C. to 39° C. The first conjugatable moieties each can include a first ligand attached to a respective one of the thermoresponsive polymer brushes, while the second conjugatable moieties each can include a second ligand attached to the cargo container. Before attachment of the cargo-containing entities, receptors can be non-covalently bound to the first ligands of the conjugatable polymer-grafted nanopillar array through one-to-one conjugation between the receptors and the first ligands. As the result, the first conjugatable moieties of the conjugatable polymer-grafted nanopillar array each can further include the receptor conjugated to the first ligand, and the non-covalent association between the first conjugatable moieties and the second conjugatable moieties can be achieved by conjugation between the receptors and the first ligands and between the receptors and the second ligands. In some embodiment, said non-covalently binding the receptors to the first ligands can be performed at a conjugation temperature at which the thermoresponsive polymer brushes exhibit contraction and hydrophobicity. More specifically, for the nanopillar array modified with LCST-typed thermoresponsive polymer brushes, the conjugation temperature is greater than the lower critical solution temperature of the thermoresponsive polymer brushes. For example, the conjugation temperature may be 32° C. or greater, 33° C. or greater, 34° C. or greater, 35° C. or greater, 36° C. or greater, 37° C. or greater, 38° C. or greater, or 39° C. or greater. Preferably, the conjugation temperature is within a range greater than 32° C. and less than 46° C., such as from 33° C. to 41° C., from 34° C. to 40° C., or from 35° C. to 39° C.

### Kits

Described herein are kits for practicing the methods described herein, e.g., for delivering exogenous species into cells. The kits may include the above-mentioned conjugatable polymer-grafted nanopillar array, and the above-mentioned conjugatable cargo-containing entities. For the aspects of the conjugatable polymer-grafted nanopillar array including no receptor conjugated to the first ligand, the kits may further include receptors each having a plurality of binding sites for conjugation with the first ligand and the second ligand. Additionally, the kits will typically include instructional materials disclosing means for generating the responsive platform (such as delivering the conjugatable cargo-containing entities to the conjugatable polymer-grafted nanopillar array to allow the attachment of the cargo-containing entities to the polymer-grafted nanopillar array); adhering cells on the responsive platform; triggering detachment of the tethered cargo-containing entities by external thermal stimulus; and incubating the cells after thermal stimulation for a period of e.g. 0.5 hour to 4 hours.

### Experimental and Methods

### [Liposome Preparation (Non-Loaded, Calcein and Doxorubicin-Loaded)]

Doxorubicin- and calcein-loaded liposomes were generated for different purposes. The calcein-loaded liposome was used to obtain cellular fluorescence images and to quantify cellular liposome uptake. The doxorubicin-loaded liposome was used in Cryo-EM imaging to demonstrate the liposome and cargo intactness when immobilized on the surface of p(NIPAAm-co-AEMA)-SiNP responsive nanosubstrate and also for cellular uptake study by breast cancer cells. To prepare empty liposome, chloroform solution containing 1,2-distearoyl-sn-glycero-3-phosphocholine (DSPC, 12.7 mM), cholesterol (25.8 mM), 1,2-distearoyl-sn-glycero-3-phosphocholin-polyethyline glycol-2000 (DSPC-PEG-2000, 3.6 mM) and 1,2-distearoyl-sn-glycero-3-phosphoethanolamine-N-[biotinyl (polyethylene glycol)-2000] (DSPE-PEG-2000 biotin, 0.33 mM) in the molar ratio of 45:50:4:1 was dried under reduced pressure to form a thin lipid film.

To prepare calcein-loaded liposomes, 1 ml of 50 mM calcein in 50 mM tricine buffer solution at pH 7 was added to the lipid film and vigorously shake for 30 mins for lipid film rehydration. After 10 times of freeze-thaw cycle, the suspension was then extruded 20 times using 100 nm pore size polycarbonate membrane filter at 60° C. using Avanti Mini-extruder to generate calcein-loaded liposome. To prepare doxorubicin liposome, 1 ml of 300 mM ammonium sulphate solution was added to the lipid film and vigorously shake for 30 mins for lipid film rehydration. After times of freeze-thaw cycle, the liposome suspension was then extruded times using 100 nm pore size polycarbonate membrane filter at 60° C. using Avanti Mini-extruder to generate ammonium sulphate-loaded liposome. For doxorubicin active-loading, 5 mM doxorubicin was added into the ammonium sulphate liposome (5 mM in phospholipid concentration), heated to 65° C., and incubated for 40 minutes to complete the loading. All the liposomes were purified using CL4B column to remove unencapsulated content using buffer solution of 50 mM tricine and 100 mM NaCl. The collected liposomes solution was stored at 4° C. until used (within 1 week).

### [Solid Surface Polymerization]

Silicon nanosubstrate pillars (SiNP) chip prepared through photolithography and wet etching by using silver nitrate (>99.8%), hydrofluoric acid (48%), ethanol (>99.5) and acetone by Prof Tseng's group (diameter: 50-500 nm; height: 3 μm; pitch: 50 nm-1 μm) was submerged in a reactor containing 200 ml of toluene and 1 ml of (3-aminopropyl) triethoxysilane, and were stirred for 60 minutes at room temperature. Later, in cold DCM solvent α-Bromobutyryl bromide (10 ml) and trimethylamine (TEA,10 ml) was added dropwise and stirred overnight at room temperature. Nanosubstrate under nitrogen environment then submerged in a solution of CH₃OH—H₂O (50:50%) and followed by addition of 0.9 mmol CuBr and 1.5 mmol N, N, N,″ N″-pentamethyldiethylenetriamine (PMDETA). Later, N-isopropyl acrylamide (NIPAAm, 28.5 mmol, 95% by weight) and amino ethyl methacrylate (AEMA, 1.5 mmol, 5% by weight) were added and stirred for 8 hours for polymer grafting. Nanosubstrate pillars after polymer grafting via atomic transfer radical polymerization (ATRP) were submerged in 200 ml of N, N-dimethylformamide (DMF), 1-Ethyl-3-(3-dimethylaminopropyl) carbodiimide (EDC, 8.7 mM) and biotin (9.2 mM) and then stirred overnight at room temperature for biotin functionalization.

[Immobilization of Liposome on p(NIPAAm-Co-AEMA)-SiNP Responsive Nanosubstrate and Cryo-EM Imaging of Doxorubicin-Loaded Liposome/Nanosubstrate]

Biotinylated responsive nanosubstrate p(NIPAAm-co-AEMA)-SiNP were incubated with streptavidin (50 μg/ml) in phosphate-buffered saline (PBS) at 37° C. for 30 mins for later surface coating of biotinylated liposomes. After streptavidin incubation, the surface was washed with PBS and these nanopillars were then cut by sharp knife gently and collected in the Eppendorf tube. Biotinylated doxorubicin-loaded liposome was incubated with polymer-grafted nanosubstrate pillars for 10 mins at 37° C. and then frozen for Cryo-EM imaging. For general liposome immobilization on responsive nanosubstrate p(NIPAAm-co-AEMA)-SiNP in later cell experiment, the incubation condition remains the same without knife-cutting of the chip.

[Liposome Quantification]

To quantify liposome surface loading and thermal detachment on responsive nanosubstrate p(NIPAAm-co-AEMA)-SiNP surface, the fluorescence signal (after liposome rupture) generated by calcein-loaded liposome was used to quantify the liposome immobilization and later temperature-induced detachment on responsive nanosubstrate p(NIPAAm-co-AEMA)-SiNP surface. For liposome coating on streptavidin-functionalized responsive nanosubstrate p(NIPAAm-co-AEMA)-SiNP, the chip holder was first mounted on and closed by a patterned PDMS lid. 300 μl of tricine buffer at pH 7.4 was passed through the nanosurface with the flow rate 0.2 ml/min to wet the surface before liposome attachment. 15 μl of 50 mM calcein-loaded liposomes were injected into chip mount with the flow rate of 0.5 ml/h and incubated for 10 mins at 37° C. for liposome attachment on the nano chip surface. The surface was then washed with 300 μl tricine buffer solution again to remove non-bonded liposomes at a flow rate of 0.2 ml/min. At this stage the liposome coating on chip surface is completed. To analyze coated and the thermo-detached liposome amount, thermo detached-cycle (THC) on the chip was applied four times (4° C.-37° C.), and the surface was washed again to collect thermally detached liposomes at 4° C. with the flow rate of 1 ml/hr., and was treated by 1% Triton X-100 detergent to rupture the liposome to fluorescently quantify calcein concentration. Liposomes that were not thermally detached and hence remained on chip surface were ruptured by passing 1% Triton X-100 with flow rate 0.2 ml/min at 37° C., and the fraction were collected to fluorescently analyzed the calcein from undetached liposome. Responsive nanosubstrate without streptavidin coating was used for quantitative estimation of nonspecific interaction.

[Confocal Visualization of Calcein-Loaded Liposome Loading and Thermal Detached on Responsive Nanosubstrate p(NIPAAm-Co-AEMA)-SiNP]

To visualized liposome coated on surface before and after THC, 50 mM calcein-loaded liposomes were used. Liposomes were immobilized on the nanosurface using same procedure used for liposome quantification study. Surface was then washed three times with 300 μl tricine buffer solution to remove all non-bonded liposome at a flow rate of 0.2 ml/min. At this stage liposome coating is completed and confocal image of this surface recorded as liposome conjugated responsive nanosubstrate before thermal control detachment. Liposomes immobilized on nanosurface were detached using 4 times thermo-released cycle (THC) from 4° C.-37° C. The surface was then washed again to remove all detached liposome before recording confocal imaging as, after thermal control detachment. Confocal images were recorded at wavelength ex/em: 495/515 nm for calcein-loaded liposome coated conjugated on responsive nanosubstrate using confocal microscope.

### [Time Depended Cell Viability Study on Plain Silicon and Responsive Nanosubstrate SiNP Surface]

To determine the best suitable biological experimental condition for responsive nanosubstrate-mediated liposome delivery system, cell viability was tested. HEK-293T cells (1×10⁵) were injected and seeded onto the responsive nanosubstrate conjugated with empty-liposome. After 10 mins of cell seeding, THC were applied in order to detach the coated empty-liposome from the nanosurface. The nanosurface with cells further incubated for 0.5, 1, 2 and 4 H with 5% CO₂ atmosphere at 37° C. Similarly, plain silicon surface was utilized for the control experiments. Cell viability was tested using live/dead kit and following the provider's procedure. Briefly, 2 μM calcein-AM and 4 μM ethidium homodimer-1 (EthD-1) was prepared in DMEM media. The solution was added to the cells coated nanosurface for 15 mins incubation. Live cells emitting green fluorescence were recorded at wavelength ex/em: 494/517 nm and on the other hand, dead cells showing red fluorescence were recorded at wavelength ex/em: 517/617 nm using fluorescent microscope.

[Thermal Stimulated Delivery of Calcein-Loaded Liposome from Responsive Nanosubstrate p(NIPAAm-Co-AEMA)-SiNP Surface (Smart and Nano Substrate Effect)]

In order to carry out the cellular uptake experiments, liposome was attached on responsive nanosubstrate using biotin-streptavidin linkage. 4 mM calcein-loaded liposome was used in order to avoid fluorescence quenching effect by calcein dye. Streptavidin bounded responsive nanosubstrate was mounted on chip holder initially and then closed by PDMS top gently. 300 μl of tricine buffer (50 mM) at pH 7.4 was passed through nanosurface at a flow rate of 0.2 ml/min to wet the surface before liposome attachment. 15 μl of 4 mM calcein-loaded liposomes were injected into chip mount with the flow rate 0.5 ml/h and incubated for 10 mins at 37° C. to tethered liposome onto the nano surface. The surface was washed again with 300 μl tricine buffer solution to remove all non-bonded liposomes from the surface at a flow rate of 0.2 ml/min. HEK-293T cells (1×10⁵) were injected and seeded on to the nanosubstrate for 10 mins. THC were applied to detach the coated liposome for cellular uptake. Cells seeded responsive nanosubstrate further incubated for 0.5, 1, 2 and 4 h with DMEM and 5% CO₂ atmosphere at 37° C. After incubation, these cells were detached using trypsin-EDTA digestion and were collected to measured fluorescence intensity of calcein-loaded liposome infused cells through fluorescence-activated cell sorting (FACS). Liposome coated surface without using thermal stimulus was used in order to demonstrate the substrate effect over solution phase approach for liposome cellular uptake. On the other hand, for solution based experiments HEK-293T cells (1×10⁵) and calcein-loaded liposome were mixed in 500 μl Eppendorf tube and 4 times THC were externally provided by dipping the cell solution into 37° C. and 4° C. cold water for 1 min each cycle before incubating for 0.5, 1, 2, and 4 h. For the control experiments in solution phase external THC were not applied. Incubated cells solution was diluted with 500 μl DMEM before recording the FACS histogram.

### [Visualize the Cellular Liposome Uptake Using Confocal Microscopy Imaging]

50 mM calcein-loaded liposomes were attached on responsive nanosubstrate first, 30 μl of HEK-293T cells (1×10⁵) in DMEM were injected (0.2 ml/min) and seeded on the liposomal functionalized chip surface for responsive nanosubstrate-mediated liposome delivery. In order to visualize the detail liposome uptake image, 3D confocal images technique was used for viewing the liposome infusion into the cellular cytoplasm. After 10 mins of cell seeding, THC were applied to detach the coated liposome for enhanced cellular delivery. Cells and the chip apparatuses were further incubated in 5% CO₂ atmosphere at 37° C. for 2 hr. After incubation, PDMS top was removed gently and cells were incubated with DiD cell labelling solution for 15 mins to stain the cell membrane. 3D confocal images were recorded using confocal fluorescence microscope. Wavelength ex/em: 633/647 nm was used for cell membrane fluorescence imaging and wavelength ex/em: 495/515 nm was used infused liposome imaging.

[Thermal Stimulated Detachment of Doxorubicin-Loaded Liposome from Responsive Nanosubstrate]

To demonstrate significance of the thermoresponsive polymer of responsive nanosubstrate, smart effect was examined using doxorubicin-loaded liposome on MCF-7 cells. Doxorubicin-loaded liposome was coated onto the responsive nanosubstrate using same procedure used for cellular uptake study. MCF-7 cells (1×10⁵) were injected and seeded on to the surface 10 mins before the THC applied; on the other hand, for the control experiment THC was not provided. Cells were further incubated for 0.5, 1, 2 and 4 h with Dulbecco's Modified Eagle's Medium (DMEM) and 5% CO₂ atmosphere at 37° C. After incubation PDMS top was removed gently and cell viability was tested using live/dead assay by following the provider's procedure. Briefly, 2 μM calcein-AM and 4 μM ethidium homodimer-1 (EthD-1) was prepared in DMEM media. The solution was added to the cells coated nanosurface for 15 mins incubation. Live cells emitting green fluorescence were recorded at wavelength ex/em: 494/517 nm and on the other hand, dead cells showing red fluorescence were recorded at wavelength ex/em: 517/617 nm using fluorescent microscope.

### [Responsive Nanosubstrate-Mediated Liposome Cellular Uptake Mechanism Study]

An attempt was made to investigate responsive nanosubstrate-mediated liposome uptake mechanism, HEK-293T cells were treated with different endocytic inhibitors to block the possible endocytosis pathways for liposome infusion into tethered cells. HEK-293T were seeded in 6-well plate at a density of 1×10⁵ cells per well and incubated for 21 h with DMEM, 5% CO₂ at 37° C. The culture media was removed after incubation and cells were once again incubated with 1 ml of endocytic inhibitors as chlorpromazine (10 μM), sodium azide (0.1% w/v in medium), genistein (200 μM), wortamannin (50 nM), nocodazole (33 μM) for 1 h at 37° C. Further, cells were washed with PBS, trypsinized, centrifuged and injected into liposome coated on nanosubstrate with the flow rate of 0.2 ml/min and seeded for 10 mins. THC were applied to detach calcein-loaded liposome for cellular uptake. Nanosubstrate coated with cells were further incubated for 4 h with 5% CO₂ at 37° C. After incubation PDMS top was removed gently and cells membrane was stained using octadecyl rhodamine B chloride (R18) dye. Confocal images were recorded at wavelength ex/em: 559/571 nm for cell membrane imaging and infused liposome imaging using ex/em: 495/515 nm using confocal microscope.

### Results and Discussion

[SiNP Surface Grafting by Biotinylated-Thermoresponsive Nanosubstrate p(NIPAAm-Co-AEMA)]

Nanopillars after responsive nanosubstrate grafting displayed alteration in its surface properties such as, hydrophilicity and nanopillars thickness. The variations in water contact angle measurement and nanopillar thickness were recorded and analyses by water contact angle measurement (WCA) and scanning electron microscopy (SEM) respectively.

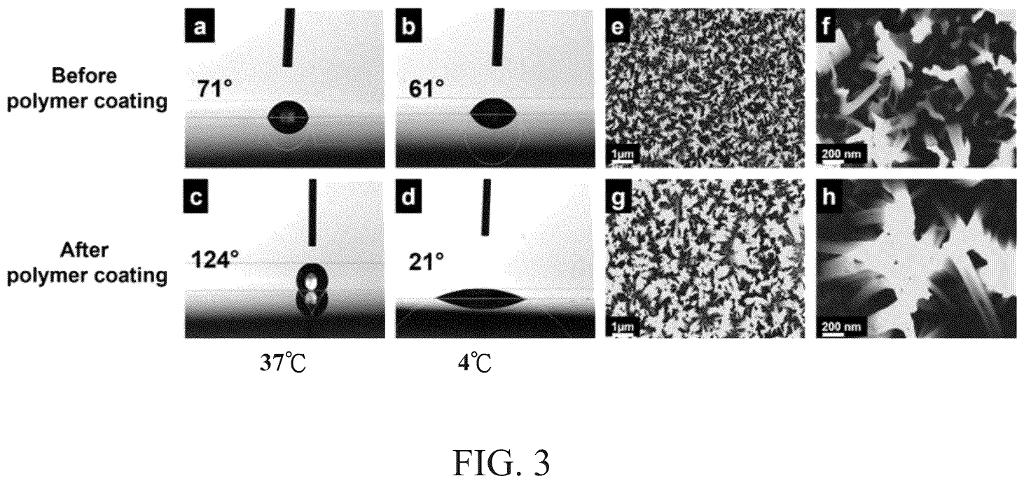

FIG. 3-a, b and c, d shows the measurement of WCA before and after responsive polymer grafting at 4° C. and 37° C. A drastic change in contact angle before and after surface polymer grafting was observed. WCA was measured as 71° at 37° C. to 61° at 4° C. (FIG. 3-a, b) for pillars before polymer grafting whereas, surface after polymer grafting shows WCA as 124° at 37° C. to 21° Pat 4° C. (FIG. 3-c, d). These changes in contact angle not only confirmed successful polymer grafting but also the thermoresponsive nature of the grafted polymer on nanopillars. Also, SEM images display an aggregated morphology of responsive polymer grafted nanopillars (FIG. 3-g, h) on comparing surface without (FIG. 3-e, f) polymer this morphology change was significant. The SEM images provide an extra evidence for confirmed biotinylated-p(NIPAAm-co-AEMA) co-polymer grafting on the surface of the nanopillars.

### [Affirmation of Liposome Preparation and Calcein, Doxorubicin Loading by Cryo-EM and Dynamic Light Scattering]

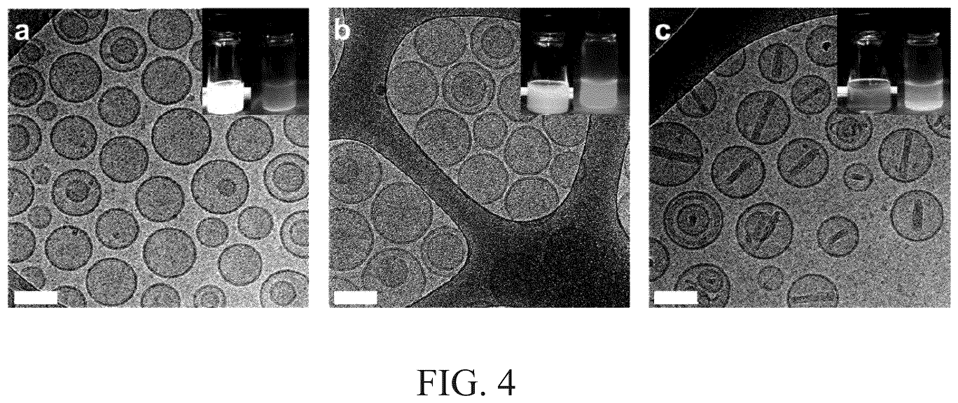

Cryo-EM imaging (FIG. 4-a, b, and c) was performed to verify liposome morphology and loading of calcein dye and doxorubicin. All liposomes images show round-shape lipid membrane projection with uniformly distributed size around 100 nm, without any significant electron density difference in the liposome aqueous core suggesting calcein dye is solubilized and does not form crystal or solid structure inside the liposomes. Except needle-like crystals were seen in the doxorubicin-loaded liposome (FIG. 4-c), which confirmed efficient doxorubicin loading. The fluorescence images of liposomes loaded with calcein and doxorubicin, and pictures were taken under visible (inset-left) and UV-365 nm (inset-right) light. Liposome size and zeta-potential were characterized by dynamic light scattering. The hydrodynamics diameter of the liposome was confirmed to be 134±4 nm (Table I) for all non-loaded, calcein-loaded and doxorubicin-loaded liposomes. The zeta-potentials are −3.1±0.2 mV illustrating that prepared liposome have similar size and surface charge despite of different content.

TABLE I
Size with polydispersity index (PdI) and zeta potential of empty,
calcein and doxorubicin-loaded liposome were analyzed.

| Sample Name | Size (d, nm) | PdI | Zeta potential (mV) |
|---|---|---|---|
| Empty liposome | 138.1 | 0.07 | −3.1 |
| calcein-loaded liposome | 131.2 | 0.04 | −3.2 |
| doxorubicin-loaded liposome | 135.8 | 0.07 | −3.4 |

### [Confirmation of Liposome Attachment on Responsive Nanosubstrate by Cryo-EM, Attachment and Detachment by Confocal Microscopy and Quantification by Released Calcein Fluorescence Intensity]

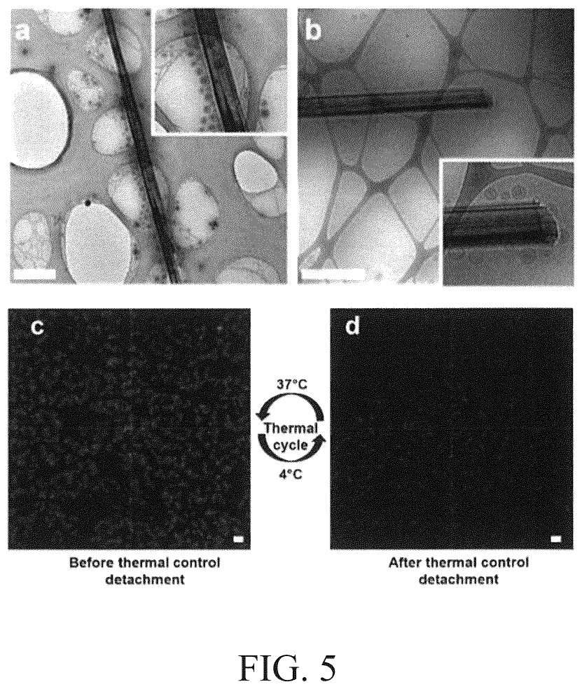

Biotin-streptavidin-biotin (B-S-B) interaction approach was used for the liposome attachments on responsive nanosubstrate surface shown in FIG. 5. For the attachment, streptavidin was coated to the biotinylated-responsive nanosurface to immobilize biotinylated liposome. In the FIG. 5, a layer of doxorubicin-loaded liposomes adhered on cuffed nanopillars were observed due to biotin-streptavidin affinity (FIG. 5-a, b). Additionally, it was observed these liposomes display no leakage of loaded doxorubicin when in contact with responsive nanosubstrate which suggests this polymer grafted nanosubstrate is compatible (softer) for liposome coating. Moreover, to observe liposome detachment after thermal stimulus, calcein-loaded liposome was attached on polymer coated nanopillars via B-S-B approach (FIG. 5-c) and after 4 cycle of thermal stimulus (FIG. 5-d) a decrement in the green fluorescence of calcein were observed after applying THC, which suggests that most of the immobilized liposome were detached from the nanosurface ensuring the significance of thermoresponsive polymer grafting.

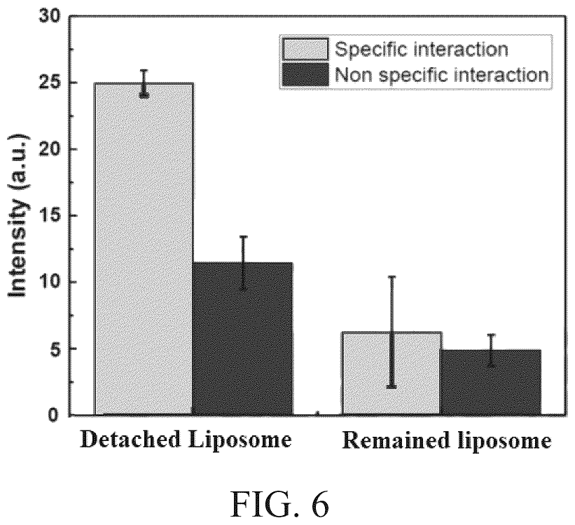

The total amount of attached calcein-loaded liposome was calculated by back calculating the amount of liposome detached and remained on responsive nanosubstrate after thermal stimulus for both types of interactions (FIG. 6). Fluorescence intensity of calcein-loaded liposome as 31 a.u. in case of specific interaction and control as 15 a.u. which is 50% less than specific interaction were observed. This shows that even without having streptavidin coating on responsive nanosubstrate some amount of liposomes still can be attached to the control surface. It is assumed this interaction might be caused due to hydrogen bonding or van der wall forces of interaction between thermoresponsive polymer and lipids. Similarly, fluorescence intensity of thermo-detached liposomes as 28 a.u. and 12 a.u. for specific interaction (90% of total attached) and control (80% of total attached) respectively were observed. Moreover, the intensity for liposomes remained were calculated as 2.5 a.u. and 2.9 a.u. (10% and 20% of total attached) for specific and control interactions, respectively. The quantification results prove that the modified responsive nanosubstrate can not only attached liposome via B-S-B approach but also can significantly detached 90% of the attached liposome upon thermal stimulation.

### [Optimum Cell Incubation Time for Responsive Nanosubstrate-Mediated Delivery]

The cell viable time on SiNP surface was an important factor to determine the best incubation condition for SiNP nano injection. HEK-293T cells were seeded on empty-liposome coated responsive nanosubstrate and incubated for 0.5, 1, 2 and 4 h, and cell viability was evaluated using live and dead assay. Plain silicon surface was employed as control.

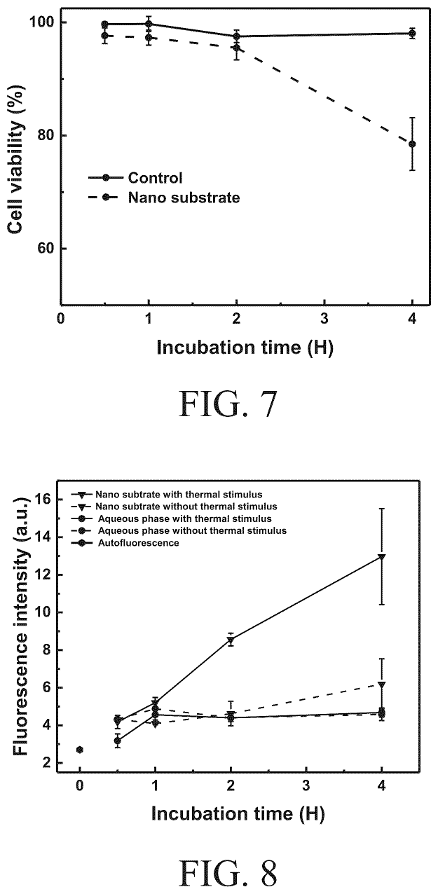

The quantification result (FIG. 7) exhibits on responsive nanosubstrate 90% were alive until 2 h as well as after 4 h incubation almost 80% cell viability was observed. On the other hand, almost 100% cell viability on plain silicon surface was recorded when incubated from 0.5 to 4 h. The results suggest that this developed responsive nanosubstrate could be adequate for liposome delivery when incubated within 4 h. Hence, all cellular experiments were conducted within 4 h incubation on responsive nanosubstrate surface.

### [Liposome Cellular Delivery Assisted by Responsive Nanosubstrate (Smart and Nano Substrate Effect)]

Cellular uptake efficiency of responsive nanosubstrate-conjugated liposome was evaluated and quantitatively compared with the free liposome in buffer using a flow cytometer (FIG. 8). Cells were incubated on responsive nanosubstrate conjugated with calcein-loaded liposomes with and without applying thermal stimulus while solution mixture of cells and free calcein-loaded liposome were utilized as solution phase approach and non-treated cells were used for auto fluorescence intensity.

Responsive nanosubstrate-mediated delivery system shows a linear increment in fluorescence intensity from 4.18 to 12.97 a.u. (triangle, solid line) when cells incubated for 0.5 to 4 h respectively. On comparing with non-treated cells (hexagon), a 10 times higher fluorescence intensity of calcein was calculated after 4 h incubation when thermal stimulus was applied. Similarly, more than 8 times higher fluorescence intensity was analyzed against solution-phase approach with (circle, solid line) and without (circle, dashed line) using an external stimulus. These observed results suggest that responsive nanosubstrate surface, not only provide a nano surface for liposome and cell to conjugate but also to generate a high local concentration of liposome on applying thermal stimulus (smart effect). However, after comparing the fluorescence intensity of non-treated cells, 3 times higher fluorescence intensity of calcein infused cells was observed for the nano surface without THC. Moreover, only 1.5 times higher intensity was recorded after comparing with the solution phase approach which suggests the significance of nano surface over the solution phase. In the case of solution-phase approach using with and without THC, a marginal increment in fluorescence intensity of calcein was observed when suspension mixture cells and calcein-loaded liposomes were incubated from 0.5 to 4 h. This low uptake was attributed to a poor internalization of the calcein-loaded liposome since no substrate was used for the liposome and cell immobilization. These data clearly show that responsive nanosubstrate mediated liposome delivery can promote liposome cellular uptake significantly, making the modified nanosystem a potential candidate for substrate assisted liposome delivery in vitro.

### [Confocal Microscopy Image of Responsive Nanosubstrate Mediated Liposome Infusion to Seeded Cells]

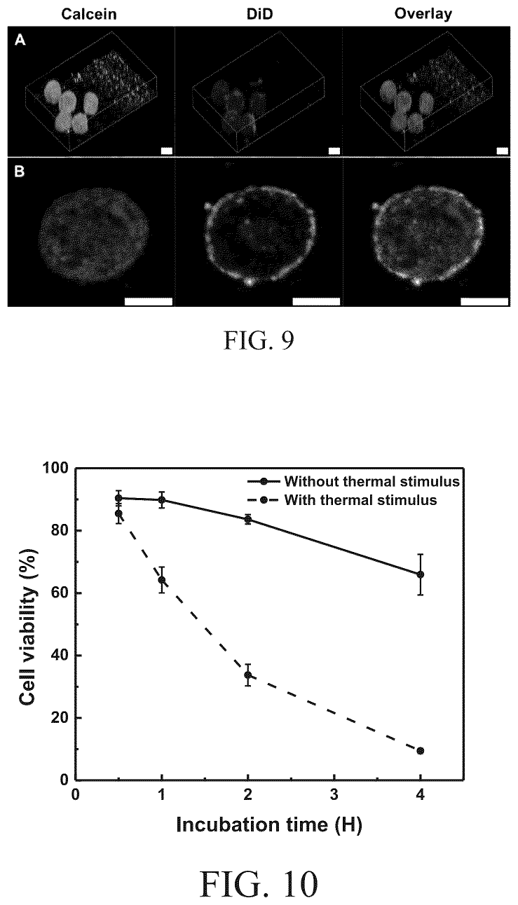

To clearly visualize cellular liposome uptake, cells on liposome conjugated responsive nanosubstrate after thermal stimulus was observed using vertical confocal microscopy. 3D reconstructed images of HEK-293T (FIG. 9-A) display green fluorescence of calcein-loaded liposome after incubation and thermal stimulus. Conjugated aggregated liposome can be observed on nanopillars where no cell attached. A centered Z-section single—cell confocal image (FIG. 9-B) was recorded which clearly shows that the green fluorescence signal originating only from the cell interior, which indicates an effective liposome infusion to the cells cytoplasm. This finding suggests responsive nanosubstrate mediated liposome delivery system can enhance the liposome cellular uptake efficiently.

### [Using Doxorubicin-Loaded Liposome to Demonstrate Cargo Loaded Liposome Delivery Via Responsive Nano Substrate]

MCF-7 cells were incubated on responsive nanosubstrate conjugated with doxorubicin-loaded liposome, using with and without thermal stimulus for 0.5 to 4 h incubation. Cell viability was evaluated using live and dead assay.

After applying THC, a linear viability decrement was observed from 0.5 to 4 h incubation (FIG. 10). With thermal stimulus, 85, 64, 33 and 10% cell viabilities were quantified for 0.5, 1, 2 and 4 h incubation respectively (FIG. 10) which suggests a high local concentration of detached doxorubicin-loaded liposomes. On the contrary, without using thermal stimulus MCF-7 cells showed cell viability up to 80% after 2 h incubation and 65% cell viability after 4 h incubation (FIG. 10). It was believed this decrement in viability could be an effect of the low local concentration of doxorubicin-loaded liposomes. These results illustrate that the detachment of doxorubicin-loaded liposome was achieved exclusively via thermal stimulus that leads to the high local concentration of doxorubicin for cellular uptake causing more cell death which further, highlights the smart effect of the nanosystem.

### [Cellular Uptake Mechanism of Responsive Nanosubstrate Mediated Liposome Delivery]

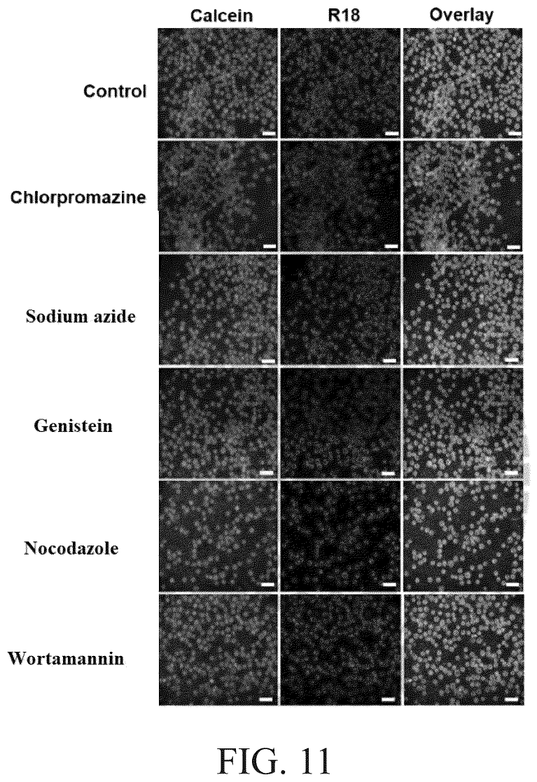

The knowledge of the cellular uptake mechanism is important to improve the nano substrate mediated delivery. To identify and resolve liposome cellular uptake pathways, such as endocytosis, membrane fusion, or direct membrane translocation, cells were pre-incubated with different endocytic inhibitors such as chlorpromazine (inhibits clathrin-mediated endocytosis), sodium azide (inhibits mitochondrial ATP production), genistein (inhibits caveola-dependent endocytosis), nocodazole (inhibits microtubule cytoskeleton) and wortamannin (inhibits PI3-kinase and micropinocytosis) to block each endocytosis uptake pathways. Cells without endocytic inhibitor pre-incubation were used as control. HEK-293T were incubated with calcein liposome-conjugated with responsive nanosubstrate surface for 4 h after THC for cellular liposome uptake. The cell membrane was further stained using R18 fluorescent dye before confocal imaging (FIG. 11).

After comparing with control experiments, HEK-293T cells display no significant drop of calcein intensity after pre-incubated with different endocytosis inhibitors suggesting endocytosis independent uptake pathway. Based on these observed results it can be concluded that the modified responsive nanosubstrate might assist liposome to migrate into the adhered cell via membrane fusion or/and direct translocation through cell membrane perturbation could be the possible delivery mechanisms.

### CONCLUSION

The platform integrating thermoresponsive polymer and liposome is an innovative approach for a controlled and efficient delivery that has the potential for the wide range of biomolecules into the nanopillar (adhered) tethered cells in vitro. The nanopillars surface modified with thermoresponsive polymer enables thermal control release of liposome to generate high local concentration for adhered cells. It has been demonstrated that liposome can not only attached on the solid surface without their shape deformation but can also be released via thermal stimuli.

Moreover, this newly developed surface equipped with liposome was used for efficient in vitro delivery in the field of nanoscience. Along with the controlled release, the system can achieve 8-fold higher liposome uptake efficiency. This newly created “nano-to-nano” delivery system could be used to deliver not only drug or dye but also various biomolecular species such as plasmid DNA, siRNA to the cells in vitro, and thus suitable for tissue, biological engineering, and cell engineering/reprogramming Further, the system could be used as a cell-based assay to study the cellular behavior cultured onto a pseudo-3-dimensional nanoenvironment, and can also be employed as a drug screening platform by loading different drug molecules into liposomes and swiftly deliver to tethered cells bypassing the endocytosis pathway in vitro.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude to indicate that the value described is within a reasonable expected range of values. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the

What is claimed is:

1. A responsive platform, comprising:
a polymer-grafted nanopillar array with thermoresponsive polymer brushes grafted onto surfaces of nanopillars;
cargo-containing entities each including a cargo container and a cargo in the cargo container;
first conjugatable moieties attached to the thermoresponsive polymer brushes; and
second conjugatable moieties each attached to the cargo container of a respective one of the cargo-containing entities, wherein the cargo-containing entities are attached to the thermoresponsive polymer brushes through non-covalent association between the first conjugatable moieties and the second conjugatable moieties.

2. The responsive platform of claim 1, wherein the cargo-containing entities are configured to allow for uptake by cells upon thermal stimulated detachment of the cargo-containing entities.

3. The responsive platform of claim 1, wherein the first conjugatable moieties each include a first ligand attached to a respective one of the thermoresponsive polymer brushes and a receptor conjugated with the first ligand, and the second conjugatable moieties each includes a second ligand attached to the cargo container and conjugated with the receptor.

4. The responsive platform of claim 3, wherein the first ligand and the second ligand each independently include a biotin-based group, and the receptor includes an avidin group, NeutrAvidin group, or streptavidin group.

5. The responsive platform of claim 1, wherein the thermoresponsive polymer brushes are configured to undergo hydrophobic—hydrophilic conversion under thermal stimulus.

6. The responsive platform of claim 1, wherein the thermoresponsive polymer brushes each include a polymer chain which has a lower critical solution temperature.

7. The responsive platform of claim 6, wherein the polymer chain exhibits expansion at a temperature below the lower critical solution temperature to cause detachment of the cargo-containing entities.

8. The responsive platform of claim 1, wherein the thermoresponsive polymer brushes are made of PNIPAAm-based polymers.

9. The responsive platform of claim 1, wherein the thermoresponsive polymer brushes each include a repeat unit represented by the following formula (I):

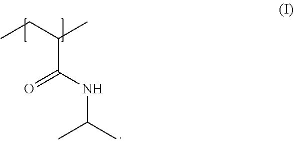

10. The responsive platform of claim 9, wherein the thermoresponsive polymer brushes each further include another repeat unit represented by the following formula (II): 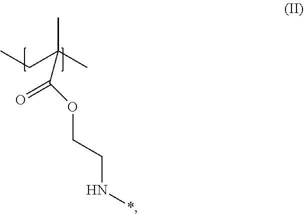
wherein the symbol * is an attachment site for the first conjugatable moiety.

11. The responsive platform of claim 1, wherein the cargo container is liposome.

12. The responsive platform of claim 1, wherein the nanopillars are silicon nanopillars.

13. A cellular delivery kit, comprising:
a conjugatable polymer-grafted nanopillar array with thermoresponsive polymer brushes grafted onto surfaces of nanopillars and first conjugatable moieties attached to the thermoresponsive polymer brushes; and
conjugatable cargo-containing entities each with a second conjugatable moiety attached to a cargo container of a cargo-containing entity, wherein the second conjugatable moiety is configured to allow non-covalent association with a respective one of the first conjugatable moieties.

14. The cellular delivery kit of claim 13, wherein the first conjugatable moieties each include a first ligand attached to a respective one of the thermoresponsive polymer brushes and a receptor conjugated with the first ligand, and the second conjugatable moiety includes a second ligand attached to the cargo container and conjugatable with the receptor.

15. The cellular delivery kit of claim 14, wherein the first ligand and the second ligand each independently include a biotin-based group, and the receptor includes an avidin moiety, NeutrAvidin moiety, or streptavidin group.

16. The cellular delivery kit of claim 13, further comprising receptors, wherein:
the first conjugatable moieties each include a first ligand attached to a respective one of the thermoresponsive polymer brushes;
the second conjugatable moiety includes a second ligand attached to the cargo container; and
the receptors each include a plurality of binding sites for conjugation with the first ligand and the second ligand.

17. The cellular delivery kit of claim 16, wherein the first ligand and the second ligand each independently include a biotin-based group, and the receptors each include an avidin group, NeutrAvidin group, or streptavidin group.

18. The cellular delivery kit of claim 13, wherein the cargo-containing entity is configured to allow for uptake by cells.

19. The cellular delivery kit of claim 13, wherein the thermoresponsive polymer brushes are configured to undergo hydrophobic-hydrophilic conversion under thermal stimulus.

20. The cellular delivery kit of claim 13, wherein the thermoresponsive polymer brushes each include a polymer chain which exhibits a lower critical solution temperature.

21. The cellular delivery kit of claim 20, wherein the polymer chain exhibits expansion at a temperature below the lower critical solution temperature to hinder non-covalent association between the conjugatable polymer-grafted nanopillar array and the conjugatable cargo-containing entities.

22. The cellular delivery kit of claim 13, wherein the thermoresponsive polymer brushes are made of PNIPAAm-based polymers.

23. The cellular delivery kit of claim 13, wherein the thermoresponsive polymer brushes each include a repeat unit represented by the following formula (I):

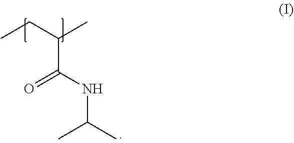

24. The cellular delivery kit of claim 23, wherein the thermoresponsive polymer brushes each further include another repeat unit represented by the following formula (II): 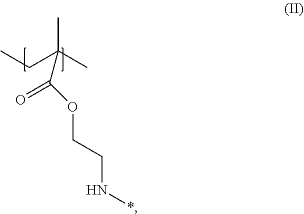
wherein the symbol * is an attachment site for the first conjugatable moiety.

25. The cellular delivery kit of claim 13, wherein the cargo container is liposome.

26. The cellular delivery kit of claim 13, wherein the nanopillars are silicon nanopillars.

27. A cellular delivery method, comprising:
providing a responsive platform which includes a polymer-grafted nanopillar array with thermoresponsive polymer brushes grafted onto surfaces of nanopillars, cargo-containing entities each including a cargo container and a cargo in the cargo container, first conjugatable moieties attached to the thermoresponsive polymer brushes, and second conjugatable moieties each attached to the cargo container of a respective one of the cargo-containing entities, wherein the cargo-containing entities are attached to the thermoresponsive polymer brushes through non-covalent association between the first conjugatable moieties and the second conjugatable moieties;
seeding cells on the responsive platform; and
applying thermal stimulus to the responsive platform to cause detachment of the cargo-containing entities from the thermoresponsive polymer brushes and to allow for uptake by the cells.

28. The cellular delivery method of claim 27, wherein the step of providing the responsive platform includes:
providing a conjugatable polymer-grafted nanopillar array having the polymer-grafted nanopillar array and the first conjugatable moieties;
providing conjugatable cargo-containing entities each having the cargo-containing entity and the second conjugatable moiety; and
attaching the cargo-containing entities to the thermoresponsive polymer brushes through the non-covalent association between the first conjugatable moieties and the second conjugatable moieties.

29. The cellular delivery method of claim 28, wherein the first conjugatable moieties each include a first ligand attached to a respective one of the thermoresponsive polymer brushes and a receptor conjugated with the first ligand, and the second conjugatable moieties each include a second ligand attached to the cargo container and conjugatable with the receptor.

30. The cellular delivery method of claim 29, wherein the first ligand and the second ligand each independently include a biotin-based group, and the receptor includes an avidin group, NeutrAvidin group, or streptavidin group.

31. The cellular delivery method of claim 28, wherein:
the first conjugatable moieties each include a first ligand attached to a respective one of the thermoresponsive polymer brushes;
the second conjugatable moieties each include a second ligand attached to the cargo container;
the step of providing the responsive platform further includes delivering receptors to the conjugatable polymer-grafted nanopillar array to allow one-to-one conjugation between the receptors and the first ligands before the step of attaching the cargo-containing entities; and
the non-covalent association between the first conjugatable moieties and the second conjugatable moieties is based on conjugation between the receptors and the first ligands and between the receptors and the second ligands.

32. The cellular delivery method of claim 31, wherein the first ligands and the second ligands each independently include a biotin-based group, and the receptors each include an avidin group, NeutrAvidin group, or streptavidin group.

33. The cellular delivery method of claim 27, wherein the thermoresponsive polymer brushes are configured to undergo hydrophobic-hydrophilic conversion under thermal stimulus.

34. The cellular delivery method of claim 27, wherein the step of seeding the cells is performed at a first temperature at which the thermoresponsive polymer brushes exhibit contraction.

35. The cellular delivery method of claim 34, wherein the step of applying the thermal stimulus includes subjecting the thermoresponsive polymer brushes to a second temperature at which the thermoresponsive polymer brushes exhibit expansion.

36. The cellular delivery method of claim 35, wherein the first temperature is greater than a lower critical solution temperature of the thermoresponsive polymer brushes, and the second temperature is lower than a lower critical solution temperature of the thermoresponsive polymer brushes.

37. The cellular delivery method of claim 27, wherein the thermoresponsive polymer brushes are made of PNIPAAm-based polymers.

38. The cellular delivery method of claim 27, wherein the thermoresponsive polymer brushes each include a repeat unit represented by the following formula (I):

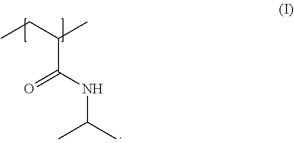

39. The cellular delivery method of claim 38, wherein the thermoresponsive polymer brushes each further include another repeat unit represented by the following formula (II): 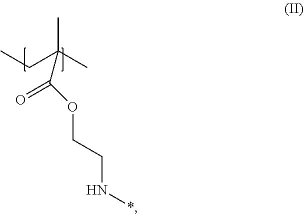
wherein the symbol * is an attachment site for the first conjugatable moiety.

40. The cellular delivery method of claim 27, wherein the cargo container is liposome.

41. The cellular delivery method of claim 27, wherein the nanopillars are silicon nanopillars.
